(19) United States
(12) Patent Application Publication
(10) Pub. No.: US 2024/0390192 A1
(43) Pub. Date: Nov. 28, 2024

(54) DISPOSABLE POUCH FOR RECEIVING AN INFLATABLE COMPRESSION SLEEVE FOR THEIR USE IN COMPRESSION THERAPY

(71) Applicant: Mego Afek AC Ltd., Kibbutz Afek (IL)

(72) Inventors: Barak HOFFER, Zikhron Ya'akov (IL); Jedidja MOY, Shorashim (IL); Neriya TSUR, Kibbutz Hahotrim (IL)

(21) Appl. No.: 18/669,967

(22) Filed: May 21, 2024

Related U.S. Application Data
(60) Provisional application No. 63/468,131, filed on May 22, 2023.

(51) Int. Cl. A61F 13/08; A61H 9/00

(52) U.S. Cl. CPC A61F 13/085; A61H 9/0078; A61H 2205/106

(57) ABSTRACT

A disposable pouch for receiving an inflatable compression sleeve, an inflatable compression sleeve, a system, and a method to use the system, for use in pneumatic compression therapy. The pouch comprising a first sheet superposed to a second sheet adjoined to form a cavity and an insertion slit for allowing reversible insertion of the inflatable compression sleeve into the cavity; a lateral edge fastening portion of the first sheet being configured to cooperate with an opposite lateral edge fastening portion of the second sheet when the pouch is wrapped in a conoid configuration for allowing releasable attachment to a limb of a user; and a flap configured to reversibly close the insertion slit and secure the inflatable compression sleeve. The inflatable compression sleeve comprising a first face superposed to a second face each made of an airtight material, being adjoined to form a plurality of adjacent inflatable pockets extending transversely across the inflatable compression sleeve, and a plurality of tubes each coupled at a first end to one of the inflatable pockets and at a second end to an inflating device connector.

### CROSS REFERENCE TO RELATED APPLICATIONS

This application claims the benefit of priority of U.S. Provisional Patent Application No. 63/468,131, filed May 22, 2023, the contents of which are incorporated herein by reference in their entirety.

### TECHNOLOGICAL FIELD

The present disclosure relates to devices and method for use in pneumatic compression therapy.

### BACKGROUND

Deep vein thrombosis (DVT} is a serious and potentially life-threatening disorder. The physiological cause of this disorder is lack of adequate blood circulation in the extremities, particularly in the lower extremities, but in some cases in the upper extremities, e.g. in the upper arm following breast cancer removal surgery. The lack of movement of venous blood can cause clots to form, which may cause blockages in the local blood vessels, or in more serious situations, may lodge in the lungs or heart and cause critical blockages which can be life-threatening.

Studies have shown that DVT can be prevented by enhancing or accelerating the return of venous blood from the extremities. A common and accepted method for accelerating venous blood return from the limbs is pneumatic compression, e.g. applied to the sole of the foot and/or the calf muscle of the leg. This form of treatment is commonly referred to as “compression therapy”, and is performed using a compression device, which feeds compressed air to a garment or “sleeve” containing one or more cells which inflate and deflate, alternately applying and releasing pressure to the extremities of the patient.

In hospitals there are many devices of this kind, and there are compression therapy usage protocols for patients who are hospitalized for operative procedures or have other risk factors for developing deep vein thrombosis. The compression therapy devices may be used 24 hours a day for the entire hospitalization period. Clinical studies have shown that the effectiveness of such devices is primarily determined by patient and staff compliance, which in turn is affected by ease of use and patient comfort. The usage of such devices is also determined by economic factors such as cost of the device and garments as opposed to pharmaceutical interventions such as heparin.

### GENERAL DESCRIPTION

The present disclosure provides systems and methods for pneumatic compression therapy which alleviate at least in part the limitations of the prior art techniques and in particular provide benefits in terms of ease of use, patient comfort and manufacturing.

In accordance with a first aspect of the presently disclosed subject matter, there is provided a disposable pouch, for receiving an inflatable compression sleeve. The disposable pouch can be used in pneumatic compression therapy. The pouch may include a first sheet superposed to a second sheet. The first and second sheets may each include a first base edge opposite a second base edge. The second base edge may be longer than the first base edge. Lateral edges may be joining the first and second base edges. The first and second sheets may be peripherally adjoined to form a cavity and an insertion slit. The insertion slit may allow reversible insertion of the inflatable compression sleeve into the cavity. The insertion slit may be formed between the second base edges of the first and second sheets. The first and second sheets may each include a lateral edge fastening portion. A lateral edge fastening portion of the first sheet may be configured to cooperate with an opposite lateral edge fastening portion of the second sheet, when the pouch is wrapped in a conoid configuration, for allowing releasable attachment to a limb of a user. The disposable pouch may further include a flap. The flap may be configured to reversibly close the insertion slit and secure the inflatable compression sleeve, when inserted into the cavity.

In addition to the above features, a disposable pouch, according to this aspect of the presently disclosed subject matter can optionally comprise one or more of features (i) to (xii) below, in any technically possible combination or permutation:
i. the pouch is configured for the first sheet to contact the limb of the user.
ii. the first sheet includes an internal layer made of an impermeable material, and a border of the cavity is defined by an inner surface of the internal layer.
iii. the first sheet includes an external layer, an outer surface of the external layer is configured to contact the limb of the user.
iv. the external layer of the first sheet is made of a water absorbing material or breathable material.
v. the external layer is adhered to the internal layer.
vi. an outer surface of the external layer is made of a material configured to releasably bind with a hook or loop fastener. Thus, the pouch may be adaptable to users of different sizes and morphologies.
vii. the lateral edge fastening portion of the second sheet comprises a hook or loop fastener strip. The hook or loop fastener strip is configured to bind with the outer surface of the external layer of the first sheet, when the pouch is wrapped into the conoid configuration.
viii. the flap comprises a band portion folding over the insertion slit.
ix. the band portion includes a second hook or loop fastener strip, configured to bind with the outer surface of the external layer of the first sheet.
x. the band portion extends from the second base portion of the first or second sheet.
xi. including a tubular opening. The tubular opening extends from the first base edges of the first and second sheets and is configured to allow passing of a tubing braid.
xii. the first and second sheets having substantially a trapezoid shape or an annulus angular section shape.

In accordance with a second aspect of the presently disclosed subject matter, there is provided a system for pneumatic compression therapy. The system includes an inflatable compression sleeve and a pouch according to the first aspect of the presently disclosed subject matter. The inflatable compression sleeve may substantially have a trapezoid shape or an annulus angular section shape. The pouch cavity may be shaped to match the inflatable compression sleeve shape, while allowing inflating and deflating the inflatable compression sleeve.

In accordance with a third aspect of the presently disclosed subject matter, there is provided a method of pneumatic compression therapy using the system of the second aspect of the presently disclosed subject matter. The method includes inserting the inflatable compression sleeve into the disposable pouch. The method includes releasably attaching the disposable pouch to a user by wrapping the disposable pouch into the conoid configuration around a limb of the user. The method may further include connecting a connector of the inflatable compression sleeve to an inflating device.

In accordance with a fourth aspect of the presently disclosed subject matter, there is provided an inflatable compression sleeve. The inflatable compression sleeve can be used in pneumatic compression therapy. The inflatable compression sleeve may substantially have a trapezoid shape or an annulus angular section shape. The inflatable compression sleeve may include a first face superposed to a second face, each made of an airtight material. The first and second faces may be adjoined to form a plurality of adjacent inflatable pockets. The inflatable pockets may extend transversely across the inflatable compression sleeve. The inflatable compression sleeve may include a plurality of tubes, each coupled at a first end to one of the inflatable pockets and at a second end to an inflating device connector. The device connector may be configured to be connected to an inflating device. The inflatable compression sleeve may further be configured to be indirectly attached to a limb of a user. This configuration may facilitate usage of the inflatable compression sleeve by providing a unidirectional configuration, thus, reducing the skill needed for operating the inflatable compression sleeve.

According to an embodiment of this aspect of the presently disclosed subject matter, the compression sleeve may be free of releasable fastening means for releasably fastening the inflatable compression sleeve to a limb of a user.

In the present application, the following terms and their derivatives may be understood in light of the below explanations:

The pouch in the present disclosure includes a first sheet and a second sheet peripherally adjoined to form a hollow cavity. The cavity may be configured to accommodate an inflatable compression sleeve.

As used herein, the terms “internal” and “external” and their derivatives may be defined with respect to a reference point of the pouch which may be in the pouch cavity e.g. a center of the pouch cavity. An internal layer may be closer to the pouch reference point than an external layer.

As used herein, the terms “inner” and “outer” may respectively be defined with reference to the pouch cavity and the environment i.e. an outside of the pouch. An inner surface may be exposed to the hollow cavity e.g. by forming a cavity wall. An outer surface may be exposed to the pouch outside environment e.g. by forming a most external pouch surface.

As used herein, “right”, “left”, “up”, down” refer to the right, left, top, bottom directions from the point of view of a reader perusing the figures. Accordingly, in FIG. 1, for example, the right direction will be understood to point along the positive y axis, while the left direction will be understood to point along the negative y axis.

As used herein, the term “trapezoid” refers to a shape having two opposite and substantially parallel base edges, wherein one base edge is longer than the other, said base edges being joined by lateral edges. It is noted that in the present disclosure, any one of the base edges and/or lateral edges of a trapezoid shape may also be curved or include one or more curves. An example is depicted in FIG. 1 where a lateral edge (left lateral edge) and a base edge (bottom base edge) of the inflatable compression sleeve include several curves. As such, the term trapezoid also encompasses an annulus angular section shape i.e. an angular section of an annulus shape.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates an inflatable compression sleeve according to the prior art;

FIG. 2 illustrates an inflatable compression sleeve according to embodiments of the present disclosure;

FIG. 3A illustrates a front view of a pouch according to embodiments of the present disclosure;

FIG. 3B illustrates a back view of the pouch of FIG. 3A;

FIG. 3C illustrates a perspective view of the pouch illustrated on FIGS. 3A-3B;

FIG. 4 illustrates a system for pneumatic compression therapy according to embodiments of the present disclosure including the pouch of FIG. 3 and the compression sleeve of FIG. 2;

FIG. 5 illustrates a system for pneumatic compression therapy according to embodiments of the present disclosure when wrapped in a conoid configuration around a limb of a user.

### DETAILED DESCRIPTION OF EMBODIMENTS

The principles, uses and implementations of the teachings herein may be better understood with reference to the accompanying description and figures. Upon perusal of the description and figures present herein, one skilled in the art will be able to implement the teachings herein without undue effort or experimentation.

In the figures, same reference numerals refer to same parts throughout. In the description and claims of the application, each of the words “comprise” “include” and “have”, and forms thereof, are not necessarily limited to members in a list with which the words may be associated.

For ease of description, a three-dimensional cartesian coordinate system (with orthogonal axes x, y, and z) is introduced. Further, the symbol ⊙ is used in the figures to represent an axis pointing “out of the page”.

Generally, the present disclosure provides a disposable pouch comprising a first sheet and a second sheet peripherally adjoined so as to form a cavity. The first and second sheets may be made of a wrappable material e.g. garment-like or film-like. The cavity is configured to accommodate an inflatable compression sleeve for use in pneumatic compression therapy. The cavity is configured to allow the inflatable compression sleeve to be accommodated in both a deflated and inflated configuration.

The pouch further comprises an insertion slit, and a flap configured to reversibly close the insertion slit. The insertion slit is configured to allow reversible insertion of the inflatable sleeve in the cavity. The insertion slit may be formed by a peripheral edge of the first sheet being partially disjoined from the second sheet. The flap is configured to allow reversibly securing the inflatable sleeve in the cavity.

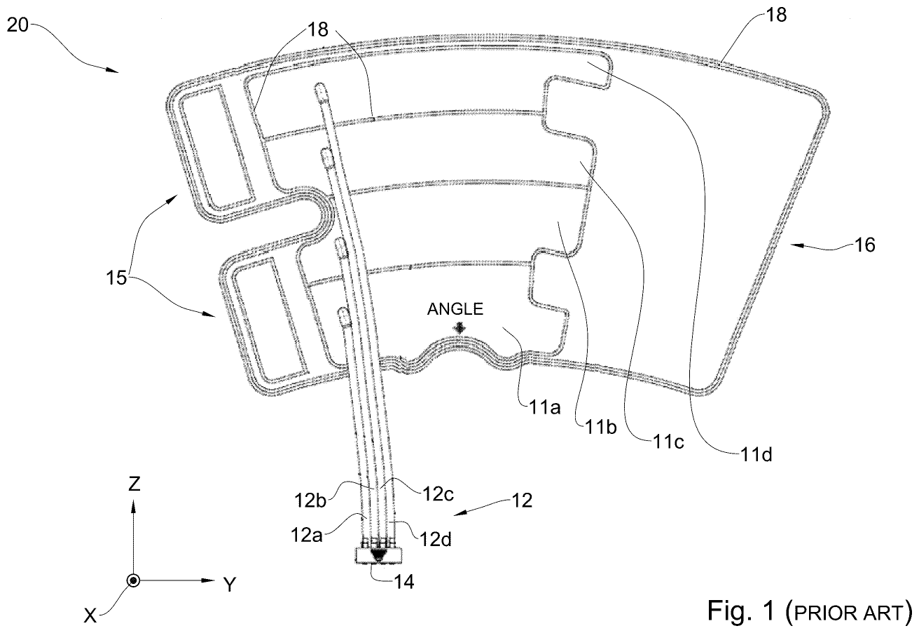

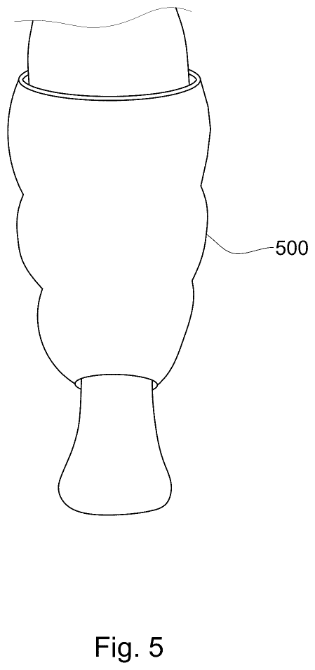

When spread out (e.g. along the yz plane as depicted in FIG. 1), the disposable pouch is substantially flat. The pouch is configured to be wrapped in a conoid configuration around a limb of a user (e.g. a leg or an arm). Preferably, the disposable pouch is substantially shaped as a trapezoid or an annulus angular section. Preferably, the cavity of the disposable pouch is also substantially shaped as a trapezoid or an annulus angular section. The pouch comprises fastening means configured to allow releasably maintaining the pouch in said conoid configuration. In use, the pouch is wrapped (i.e. rolled) in the conoid (and/or tubular) configuration around a limb of a user as illustrated in FIG. 5. The fastening means allow releasably attaching the pouch to the user limb.

Generally, the present disclosure also provides for an inflatable compression sleeve cooperating with the disposable pouch. The inflatable compression sleeve is configured to be indirectly attached to a limb of a user. The inflatable compression sleeve is free of releasable fastening means for releasably fastening the inflatable compression sleeve to a limb of a user. A shape of the inflatable compression sleeve may allow insertion into and/or match a shape of the pouch cavity. When spread out (e.g. along the yz plane as depicted in FIG. 1) and deflated, the inflatable compression sleeve is substantially flat. The inflatable compression sleeve is configured to be wrapped in a conoid configuration. The compression sleeve may be made of a wrappable airtight material. Preferably, the inflatable compression sleeve is substantially shaped as a trapezoid or an annulus angular section. In use, the inflatable compression sleeve is inserted in the cavity of the disposable pouch and the system composed of the pouch and the sleeve is wrapped in the conoid configuration around a limb of a user. The inflatable compression sleeve includes a plurality of adjacent inflatable pockets extending transversely across the inflatable compression sleeve. The compression sleeve may include a first face superposed to a second face each made of an airtight material, said first and second faces being adjoined to form a plurality of adjacent inflatable pockets extending transversely across the inflatable compression sleeve. In use, when wrapped around a limb of the user, sequentially inflating the pockets may allow improving blood circulation. The inflatable sleeve further includes a plurality of tubes configured to allow selectively inflating the inflatable pockets. The plurality of tubes may include an inflating device connector configured to be connected to an inflating device. The plurality of tubes may each be coupled at a first end to one of the inflatable pockets and at a second end to the inflating device connector.

FIG. 1 illustrates an inflatable compression sleeve 10 according to the prior art. The inflatable compression sleeve 10 includes a first face and a second face both made of an airtight wrappable material, said faces being welded together along a scam 18 forming a plurality of inflatable pockets 11a-11d each connected to an end of one of a plurality of tubes 12a-12d. The second ends of the plurality of tubes 12a-12b are coupled to an inflating device connector 14. The compression sleeve 10 further comprises two hook or loop fastener bands 15 cooperating with a fastening portion 16 of the compression sleeve to allow releasably attaching the compression sleeve 10 to a limb of a user when the compression sleeve 10 is wrapped around the limb of the user.

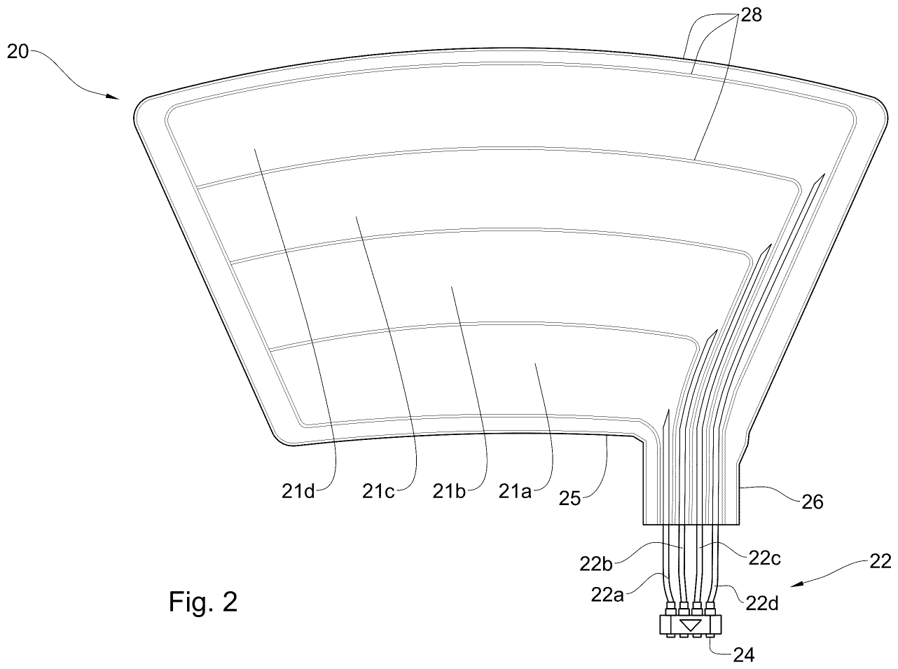

FIG. 2 illustrates an inflatable compression sleeve 20 according to some embodiments of the present disclosure. The inflatable compression sleeve 20 is configured to be indirectly attached to a limb of a user by being inserted in a disposable pouch according to the present disclosure and described in more details hereinbelow. When spread out (e.g. along the yz plane as depicted in FIG. 1) and deflated, the inflatable compression sleeve 20 is substantially flat. The inflatable compression sleeve 20 is configured to be wrapped in a conoid configuration. The inflatable compression sleeve 20 is substantially shaped as a trapezoid. The compression sleeve may be made of a wrappable airtight material. The compression sleeve may be made from a first face superposed to a second face each made of an airtight material, said first and second faces being adjoined (e.g. welded) along a scam 28 to form a plurality of adjacent inflatable pockets 21a-21d extending transversely across the inflatable compression sleeve 20. The first and second faces may have trapezoid shapes. The inflatable pockets 21a-21d may extend laterally from one lateral edge to the other lateral edge of the compression sleeve 20. The inflatable pockets 21a-21d may each have a trapezoid shape of increasing base length.

Each inflatable pocket 21a-21d may include an air inlet. The inflatable sleeve 20 further includes a plurality of tubes 22a-22d. The plurality of tubes 22a-22d form a tubing braid 22. Each tube 22a-22d may have an end coupled to a corresponding air inlet of the plurality of inflatable pockets 21a-21d to allow controllably inflating and deflating each pocket by blowing or sucking air in the tubes 22a-22d. The inflatable pockets 21a-21d can be selectively inflated and/or deflated. The air inlets may be configured so that the plurality of tubes 22a-22d connected thereto extend jointly from an extremity of a short base edge of the compression sleeve 20. The plurality of tubes 22a-22d may extend substantially perpendicular to the short base edge of the compression sleeve 20. A tubing portion 26 may projects from a short base edge of the compression sleeve and be configured to jointly guide the plurality tubes 22a-22d. Another end of the plurality of tubes 22a-22d is coupled to an inflating device connector 24.

The inflatable compression sleeve is free of releasable fastening means for releasably fastening the inflatable compression sleeve to a limb of a user. Indeed, in contrast with the inflatable compression sleeve 10 according to the prior art, the inflatable compression sleeve 20 does not include any fastener bands. This allows for more of the compression sleeve to be available for forming the inflatable pockets 21a-21d and thereby improves the sleeve adaptability.

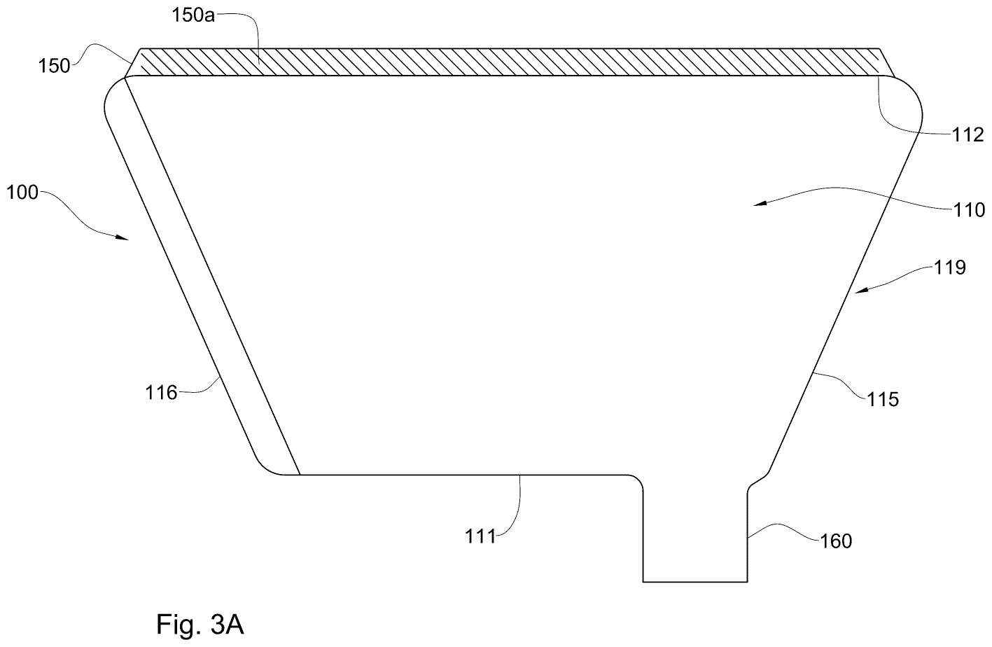

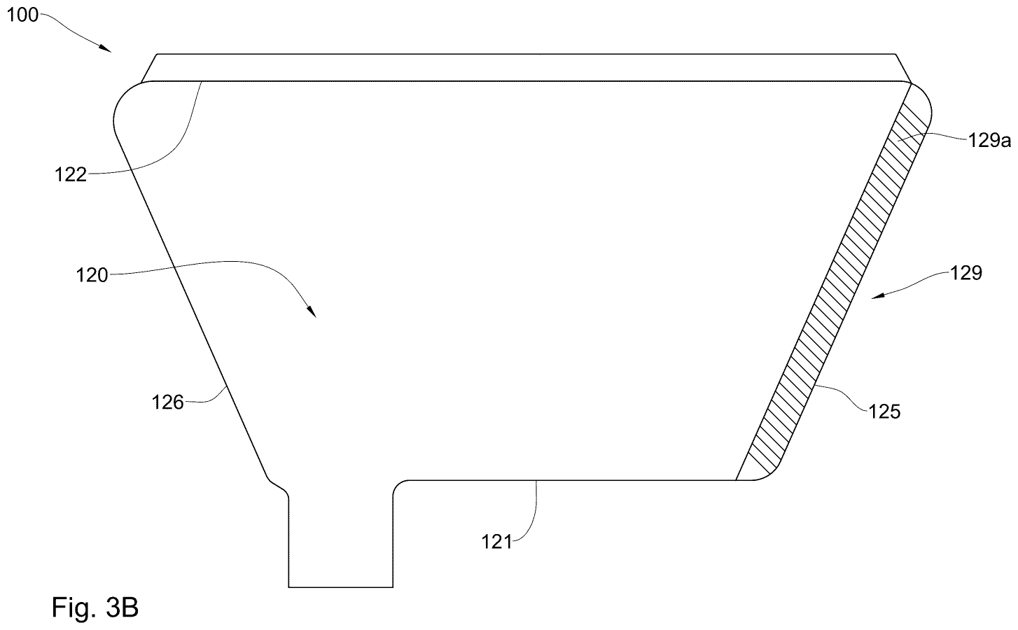

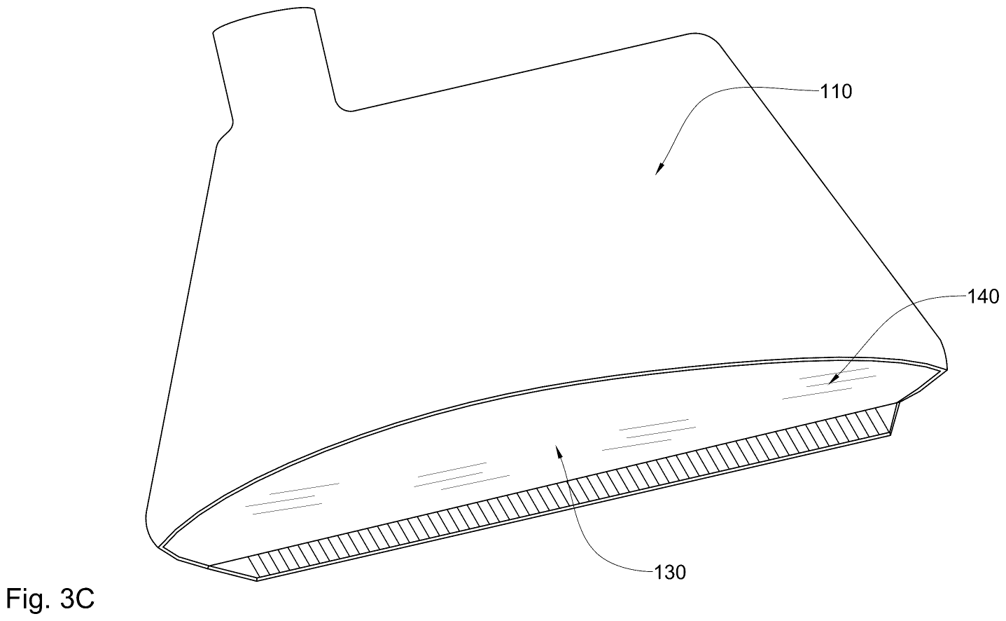

The pouch of the present disclosure can be used with a compression sleeve of the kind shown in FIG. 1 or FIG. 2, or any other suitable kind, and it is now described with reference to FIGS. 3A-3C which respectively illustrate a front, back and perspective views of the pouch 100 according to some embodiments of the present disclosure. As explained above, the present disclosure provides a disposable pouch 100 comprising a first sheet 110 and a second sheet 120 peripherally adjoined so as to form a cavity 130. The cavity 130 is configured to allow the inflatable compression sleeve (e.g. the compression sleeve 20 illustrated in FIG. 2 previously described) to be accommodated in both a deflated and inflated configuration.

The pouch 100 further comprises an insertion slit 140, and a flap 150 configured to reversibly close the insertion slit 140. The insertion slit 140 is configured to allow reversible insertion of the inflatable sleeve in the cavity 130. The flap 150 is configured to allow reversibly securing the inflatable sleeve in the cavity 130.

The pouch 100 comprises a first sheet 110 laid over a second sheet 120 made of thin wrappable materials (e.g. garment-like). The first and second sheets 110, 120 have overlapping trapezoid shapes. The first and second sheets 110, 120 each include a first base edge 111, 121 opposite a second base edge 112, 122. The second base 112, 122 edge is longer than the first base edge 111, 121. The first and second sheets 110, 120 further include respectively lateral edges 115, 116 and 125, 126 joining the first base edges and the second base edges of the first and second sheets 110, 120. The first and second sheets 110, 120 are peripherally adjoined to form the cavity 130 and the insertion slit 140. The first and second sheets 110, 120 may for example be adjoined along their edges by sewing, gluing, stapling, adhesive, or the like. The insertion slit 140 may be formed between the second base edges 112, 122, of the first and second sheets 110, 120 by at least partially not adjoining (or disjoining) said second base edges 112, 122.

The pouch 100 is shaped as a trapezoid and can be easily wrapped in a conoid configuration around a limb of a user as illustrated on FIG. 5. The conoid configuration notably allows folding of the pouch in a conoid configuration with a generally low overlap between the bottom right and bottom left portions of the pouch in comparison with a rectangular shape. In use, this is useful during inflation of the inflatable compression sleeve which is releasably embedded in the pouch cavity to avoid over-pressure in the overlapping areas. Further, the conoid configuration improves adaptability on upper leg portions of the user. In order to accommodate inflatable compression sleeves with shapes similar to the inflatable compression sleeves illustrated in FIGS. 1 and 2, the cavity 130 of the disposable pouch 100 is also substantially shaped as a trapezoid.

Further, the disposable pouch 100 comprises fastening means configured to allow releasably maintaining the pouch in said conoid configuration. The fastening means allow releasably attaching the pouch to the user limb. The fastening means include a first lateral edge fastening portion 119 on the outer surface of the first sheet 110 cooperating with an opposite second lateral edge fastening portion 129 on the outer surface of the second sheet 120 when the pouch is wrapped in a conoid configuration. For example, the first and second lateral edge fastening portions may include a hook and loop attachment mechanism. Hook and loop attachment mechanisms such as Velcro® typically include two components such as two strips which are attached to the opposing surfaces to be fastened. The first component features tiny hooks and the second features smaller loops. When the two components are pressed together, the hooks catch in the loops and the two surfaces bind temporarily. In other embodiments, the first and second lateral edge fastening portions 119, 129 may include hook, button, snaps, zippers, latches, buckles, or the like. In some embodiments, at least the first lateral edge fastening portion 119—and advantageously the entire outer surface of the first sheet 110—comprises (or is made of) a material configured to releasably bind with a hook or loop fastener. Further, at least the second lateral edge fastening portion 129—and in some embodiments, the entire outer surface of the second sheet—comprises (or is made of) a material cooperating with said first lateral edge fastening portion. In the embodiment illustrated, the pouch 100 includes a hook or loop fastening strip 129a sewed on the second lateral edge fastening portion 129 and the outer surface of the first sheet 110 is entirely made of a material cooperating with the strip 129a to allow releasable attachment. In particular, the first hoop or loop fastener strip 129a can be at least partially sewn to the second lateral edge fastening portion 129 while sowing of the first and second sheets 110, 120 i.e. a common seam adjoins the first and second sheets 110, 120 and the fastener strip 129a.

The pouch 100 may be configured for the first sheet 110 to contact the limb of the user. In particular, the first sheet 110 may comprise an internal layer made of an impermeable material. This enables to protect the compression sleeve from user's fluids such as sweat. An inner surface of the internal layer may then define the cavity 130. The first sheet 110 may also comprise an external layer adhered to the internal layer. An outer surface of the external layer may be configured to contact the limb of the user. In particular, the external layer of the first sheet 110 can be made of a water absorbing material and/or comfortable soft fabric. In the embodiments where the first sheet comprises an internal layer and an external layer, an outer surface of the external layer may be additionally configured to cooperate with the second lateral edge fastening portion 129. For example, the fastening strip 129a may be made of hook nylon, hook PE, hook PP or hook TPU and the external layer of the first sheet may be made of loop PE, loop EVA, loop PVC, or loop TPU.

The flap 150 forms a band portion folding over the insertion slit 140. The band portion may extend from the second base edge of the first or second sheet 110, 120. When folded over the insertion slit, the band portion may fully overlap over the second base edge of the first or second sheet 110, 120 so as to allow closing the insertion slit 140 like a postal envelope flap. The band portion may include securing means to enable releasably maintaining the flap folded over the insertion slit 140. For example, the band portion may include a second hoop or loop fastener strip 150a cooperating with an outer surface of the first sheet 110 to allow releasable attachment. For example, the fastening strip 150a may be made of hook nylon, hook PE, hook PP or hook TPU. For example, the second hoop or loop fastener strip 150a can be sewn to the band portion. In particular, the second hoop or loop fastener strip 150a can be at least partially sewn to the band while sowing of the first and second sheets 110, 120.

The pouch 100 may also comprise an opening to allow passing of the tubing braid 22 therethrough. In some embodiments, the pouch includes a tubular opening 160 extending perpendicularly from an extremity the first base edges 111, 121 of the first and second sheets 110, 120.

The pouch 100 of the present disclosure is also particularly easy to manufacture. In particular, attachment of the first and second sheets and of the first and second hook or loop fastener strips 129a, 150a may be performed by sewing. The number of seams necessary to perform said attachment may be less than 5. Preferably, the number of seam necessary to attach the first and second sheets and the first and second hook or loop fastener strips may be 3 or 4.

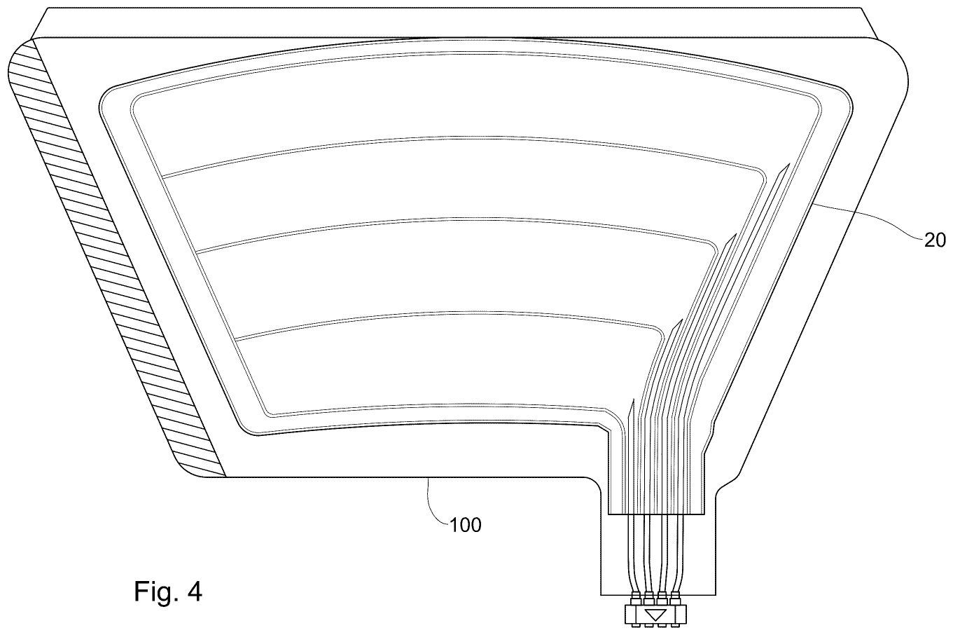

As illustrated in FIG. 4, in use, the inflatable compression sleeve 20 is inserted in the cavity 130 of the disposable pouch 100. The tubing braid 22 is positioned to exit from the tubular opening 160. As illustrated in FIG. 5, the system 500 composed of the pouch 100 and the embedded sleeve 20 is wrapped in the conoid configuration around a limb of a user. The inflating device connector 24 is then connected to an inflating device for pneumatic compression therapy.

Although certain aspects of the invention have been exemplified in the context of to treating DVT in a lower leg of a subject, it will be readily appreciated by the skilled person that these same aspects may also be of relevance in treating DVT in other limbs, other than a lower leg, such as an upper arm.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. No feature described in the context of an embodiment is to be considered an essential feature of that embodiment, unless explicitly specified as such.

Although steps of methods according to some embodiments may be described in a specific sequence, methods of the invention may comprise some or all of the described steps carried out in a different order. A method of the invention may comprise all of the steps described or only a few of the described steps. No particular step in a disclosed method is to be considered an essential step of that method, unless explicitly specified as such.

Although the invention is described in conjunction with specific embodiments thereof, it is evident that numerous alternatives, modifications and variations that are apparent to those skilled in the art may exist. Accordingly, the invention so embraces all such alternatives, modifications and variations that fall within the scope of the appended claims. It is to be understood that the invention is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the invention. Section headings are used herein to case for understanding of the specification and should not be construed as necessarily limiting.

1. A disposable pouch for receiving an inflatable compression sleeve for use in pneumatic compression therapy, the pouch comprising:
a first sheet superposed to a second sheet,
said first and second sheets each including a first base edge opposite a second base edge, the second base edge being longer than the first base edge and lateral edges joining said first and second base edges;
said first and second sheets being peripherally adjoined to form a cavity and an insertion slit for allowing reversible insertion of the inflatable compression sleeve into the cavity, the insertion slit being formed between the second base edges of the first and second sheets;
a lateral edge fastening portion of the first sheet being configured to cooperate with an opposite lateral edge fastening portion of the second sheet when the pouch is wrapped in a conoid configuration for allowing releasable attachment to a limb of a user; and
a flap configured to reversibly close the insertion slit and secure the inflatable compression sleeve when inserted into the cavity.

2. The pouch according to claim 1, wherein the pouch is configured for the first sheet to contact the limb of the user.

3. The pouch according to claim 1, the first sheet comprising an internal layer made of an impermeable material and wherein a border of the cavity is defined by an inner surface of the internal layer.

4. The pouch according to claim 1, the first sheet comprising an external layer, an outer surface of the external layer being configured to contact the limb of the user.

5. The pouch according to claim 4, wherein the external layer of the first sheet is made of a water absorbing material or breathable material.

6. The pouch according to claim 4, wherein the external layer is adhered to the internal layer.

7. The pouch according to claim 4, wherein an outer surface of the external layer is made of a material configured to releasably bind with a hook or loop fastener.

8. The pouch according to claim 7, wherein the lateral edge fastening portion of the second sheet comprise a hook or loop fastener strip configured to bind with the outer surface of the external layer of the first sheet when pouch is wrapped into the conoid configuration.

9. The pouch according to claim 1, wherein the flap comprises a band portion folding over the insertion slit.

10. The pouch according to claim 7, wherein the flap comprises a band portion folding over the insertion slit and said band portion includes a second hook or loop fastener strip configured to bind with the outer surface of the external layer of the first sheet.

11. The pouch according to claim 9, wherein the band portion extends from the second base portion of the first or second sheet.

12. The pouch according to claim 10, wherein the band portion extends from the second base portion of the first or second sheet.

13. The pouch according to claim 1, further comprising a tubular opening extending from the first base edges of the first and second sheets and configured to allow passing of a tubing braid.

14. The pouch according to claim 1, wherein the first and second sheets having substantially a trapezoid shape or an annulus angular section shape.

15. The pouch according to claim 1, constituting a part of a system for pneumatic compression therapy, said system at least when in use, comprising at least said pouch and an inflatable compression sleeve having substantially a trapezoid shape or an annulus angular section shape and the pouch cavity being shaped to match the inflatable compression sleeve shape while allowing inflating and deflating the inflatable compression sleeve.

16. A method of pneumatic compression therapy using a system comprising a pouch according to claim 1 and an inflatable compression sleeve having substantially a trapezoid shape or an annulus angular section shape and the pouch cavity being shaped to match the inflatable compression sleeve shape while allowing inflating and deflating the inflatable compression sleeve, the method comprising:
(a) inserting the inflatable compression sleeve into the disposable pouch;
(b) releasably attaching the disposable pouch to a user by wrapping the disposable pouch into the conoid configuration around a limb of the user;
(c) connecting a connector of the inflatable compression sleeve to an inflating device.

17. An inflatable compression sleeve for use in pneumatic compression therapy, the inflatable compression sleeve having substantially a trapezoid shape or an annulus angular section shape and comprising:
(a) a first face superposed to a second face each made of an airtight material, said first and second faces being adjoined to form a plurality of adjacent inflatable pockets extending transversely across the inflatable compression sleeve;
(b) a plurality of tubes each coupled at a first end to one of the inflatable pockets and at a second end to an inflating device connector configured to be connected to an inflating device;
the inflatable compression sleeve being further configured to be received within a pouch and to be attached to a limb of a user by means of said pouch.

18. The inflatable compression sleeve according to claim 17, the compression sleeve being free of releasable fastening means for releasably fastening the inflatable compression sleeve to a limb of a user.

19. The inflatable compression sleeve according to claim 17, wherein said pouch is constituted by a disposable pouch for receiving an inflatable compression sleeve for use in pneumatic compression therapy, the pouch comprising:
a first sheet superposed to a second sheet,
said first and second sheets each including a first base edge opposite a second base edge, the second base edge being longer than the first base edge and lateral edges joining said first and second base edges;
said first and second sheets being peripherally adjoined to form a cavity and an insertion slit for allowing reversible insertion of the inflatable compression sleeve into the cavity, the insertion slit being formed between the second base edges of the first and second sheets;
a lateral edge fastening portion of the first sheet being configured to cooperate with an opposite lateral edge fastening portion of the second sheet when the pouch is wrapped in a conoid configuration for allowing releasable attachment to a limb of a user; and
a flap configured to reversibly close the insertion slit and secure the inflatable compression sleeve when inserted into the cavity
